(19) United States
(12) Patent Application Publication
(10) Pub. No.: US 2020/0121296 A1
(43) Pub. Date: Apr. 23, 2020

(54) MOTION ARTIFACT SUPPRESSION IN ULTRASOUND COLOR FLOW IMAGING

(71) Applicant: EchoNous, Inc., Redmond, WA (US)

(72) Inventors: Chi Hyung Seo, Sammamish, WA (US); Ron Broad, Arlington, WA (US)

(21) Appl. No.: 16/659,021

(22) Filed: Oct. 21, 2019

Related U.S. Application Data
(60) Provisional application No. 62/748,866, filed on Oct. 22, 2018.

(51) Int. Cl. A61B 8/08; A61B 8/00; G06F 3/01

(52) U.S. Cl. CPC A61B 8/5276; G06F 3/017; A61B 8/4254; A61B 8/5207

(57) ABSTRACT

Devices, systems, and methods adjust one or more color flow parameters in response to a sensed motion of an ultrasound probe exceeding a motion threshold. One such method includes acquiring, by an ultrasound imaging device including an ultrasound probe, ultrasound image information; sensing, by a motion sensor, a motion of the ultrasound probe; determining whether the sensed motion of the ultrasound probe exceeds a motion threshold; adjusting one or more color flow parameters in response to determining that the sensed motion of the ultrasound probe exceeds the motion threshold; and generating an ultrasound color flow image based on the acquired ultrasound image information and the one or more color flow parameters.

### BACKGROUND

### Technical Field

The present disclosure pertains to ultrasound systems, and more particularly to ultrasound systems and methods for suppressing motion artifacts in an ultrasound color flow image due to motions of the ultrasound probe.

### Description of the Related Art

Color flow ultrasound imaging is an ultrasound imaging modality which is commonly used to identify the presence and direction of flow in an imaged region. In a typical color flow Doppler ultrasound imaging system, a color-coded map of Doppler shifts (color flow data) is superimposed onto a B-mode ultrasound image. The color flow data indicates flow information (e.g., blood flow information) by assigning different colors to different flow characteristics. For example, the color red is typically used to indicate flow direction toward the ultrasound transducer, while the color blue is typically used to indicate flow direction away from the ultrasound transducer. Similarly, different velocities of the flows may be indicated with different color hues or saturation.

During use, the ultrasound probe may be moved while a region of a patient is being imaged. Such movements of the ultrasound probe may be incorrectly detected as blood flow, and result in the appearance of flash artifacts (or motion artifacts) in the generated ultrasound color flow images. More particularly, the flash artifacts result from signal decorrelation caused by the probe movements and are typically narrowband and of low velocity. The artifacts appear as large patches of false flow that often covers both the imaged tissue and the true flow.

Various techniques have been implemented to suppress motion artifacts from ultrasound color flow images. One such technique involves the use of traditional Fourier-based clutter filters, which may be effective at removing motion artifacts, but also decrease the low flow sensitivity of the ultrasound color flow images. Accordingly, flows that may be of particular interest (e.g., relatively low velocity blood flows) may undesirably be filtered out of the resulting ultrasound color flow images by such techniques.

Other methods attempt to suppress flash artifacts by removing large flow excursions between successive frames. These methods assume flash artifacts are impulsive, e.g., lasting only one or few frames, which is not always true. Moreover some type of velocities such as arterial flow could also result in large excursions between successive frames, thus giving a false positive removal. Accordingly, certain flows of interest may undesirably be removed from the ultrasound color flow images. Other techniques attempt to identify flash artifacts based on the absolute energy and velocity at regions of fixed kernel size such as a line or frame. Such techniques rely on the assumption that flash artifacts and true flow do not overlap in the energy-velocity space, which is not always true. Moreover, the fixed kernel size severely limits the effectiveness of identifying flash artifacts which have a size that is highly variable.

### BRIEF SUMMARY

The present disclosure, in part, addresses a desire for ultrasound systems that are capable of suppressing color Doppler motion artifacts which cannot be removed by traditional Fourier based clutter filters without affecting blood flow sensitivity. Flash artifacts arising from tissue or probe motion are undesirable since they obscure the blood flow as well as the B mode image. The present disclosure provides systems, devices, and methods capable of identifying and removing such flash artifacts without sacrificing low flow sensitivity.

In some embodiments provided by the present disclosure, a motion sensor, such as an accelerometer and/or a gyroscope, is integrated into or otherwise coupled to an ultrasound probe to identity the motion of the probe. Motion artifacts are removed from the ultrasound color flow images based on the sensed motion of the probe. A motion sensor signal includes motion information which indicates motion of the transducer in the probe, and the motion information is provided to a color flow processor. Based on the motion information, the color flow processor determines whether the sensed motion of the probe exceeds a threshold level of the motion. If the motion of the probe exceeds the threshold, the color flow processor reduces or removes motion artifacts by adjusting one or more ultrasound processing or color flow parameters. In some embodiments, the motion artifacts may be removed or reduced by reducing their energy and/or velocity or by increasing one or more color thresholds. The motion information can also be used for further processing to reduce persistence and/or color gain when there is motion of the probe.

In some embodiments, the motion thresholding operation (e.g., determining whether the sensed motion of the probe exceeds a motion threshold) may be integrated into the motion sensor itself. In such embodiments, if the sensed motion is less than the motion threshold, the sensor signal (or motion information) is not transmitted to the color flow processor and the color flow processor does not remove or reduce motion artifacts in the generated ultrasound color flow image.

In at least one embodiment, a method is provided that includes: acquiring, by an ultrasound imaging device including an ultrasound probe, ultrasound image information; sensing, by a motion sensor, a motion of the ultrasound probe; determining whether the sensed motion of the ultrasound probe exceeds a motion threshold; adjusting one or more color flow parameters in response to determining that the sensed motion of the ultrasound probe exceeds the motion threshold; and generating an ultrasound color flow image based on the acquired ultrasound image information and the one or more color flow parameters.

In another embodiment, the present disclosure provides an ultrasound imaging device that includes an ultrasound probe. The ultrasound probe includes at least one motion sensor which, in use, senses motion of the ultrasound probe. A computer-readable memory stores color flow parameters, and a color flow processor is coupled to the motion sensor. The color flow processor, in use: receives ultrasound image information acquired by the ultrasound probe; accesses at least one of the color flow parameters stored in the computer-readable memory; receives, from the at least one motion sensor, motion information indicative of a sensed motion of the ultrasound probe; adjusts the at least one of the color flow parameters in response to the sensed motion of the ultrasound probe exceeding a motion threshold; and generates an ultrasound image based on the ultrasound image information and the at least one of the color flow parameters.

In yet another embodiment, the present disclosure provides a system that includes an ultrasound probe configured to acquire ultrasound image information. At least one motion sensor is coupled to the ultrasound probe and is configured to sense a motion of the ultrasound probe. A computing device is coupled to the ultrasound probe and to the at least one motion sensor. The computing device includes a color flow processor configured to: determine whether the sensed motion of the ultrasound probe exceeds a motion threshold; adjust one or more color flow parameters in response to determining that the sensed motion of the ultrasound probe exceeds the motion threshold; and generate an ultrasound color flow image based on the acquired ultrasound image information and the one or more color flow parameters.

### BRIEF DESCRIPTION OF THE SEVERAL VIEWS OF THE DRAWINGS

FIG. 1 is a schematic illustration of an ultrasound imaging device, in accordance with one or more embodiments of the present disclosure.

FIG. 2 is a block diagram illustrating components of the ultrasound imaging device, in accordance with one or more embodiments of the present disclosure.

FIG. 3 is a flow diagram illustrating a method, in accordance with one or more embodiments of the present disclosure.

### DETAILED DESCRIPTION

An ultrasound imaging device may include a handheld computing device and an ultrasound probe that receives ultrasound imaging signals, e.g., ultrasound echo signals returning from a target structure in response to transmission of an ultrasound pulse or other ultrasound transmission signal. The ultrasound probe includes a motion sensor which is capable of sensing motion of the probe, for example, during acquisition of ultrasound image information. Motion information sensed by the motion sensor may be compared with a motion threshold to determine whether the motions of the probe exceed the motion threshold. In response to determining that the motion information exceeds the motion threshold, one or more color flow parameters may be adjusted. The color flow parameters may be adjusted, for example, by a color flow processor which may be positioned within the computing device. The color flow parameters may be adjusted to filter out or otherwise reduce or remove motion artifacts from ultrasound color flow images that are generated based on the ultrasound image information and on the color flow parameters. For example, if the probe motions are significant enough to otherwise cause flash artifacts in the ultrasound color flow images, such motions are sensed by the motion sensor and exceed the motion threshold. Accordingly, one or more color flow parameters, such as a color flow (or velocity) parameter, a power (or energy) parameter, a color gain parameter, a persistence parameter, or the like, are adjusted to remove the motion artifacts from the generated ultrasound color flow image. In various embodiments of the present disclosure, motion information may be considered exceeding a motion threshold in a positive or negative direction, i.e., having a value above a threshold value in a positive direction or having a value below the threshold value in a negative direction, depending on the nature of the motion information and the threshold comparison that is made.

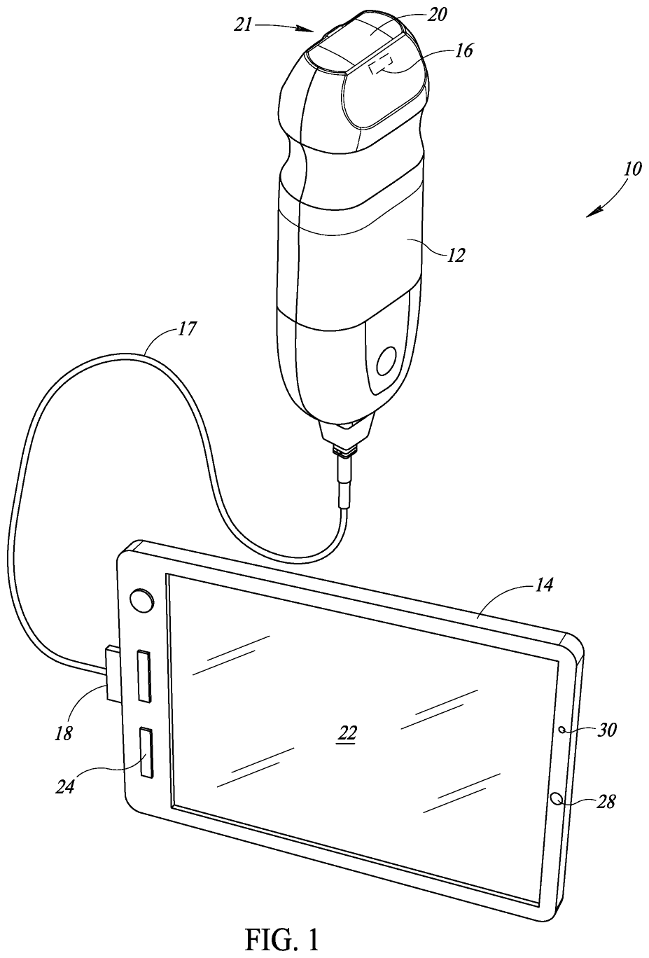

FIG. 1 is a schematic illustration of an ultrasound imaging device 10 (referred to herein as “ultrasound device” 10), in accordance with one or more embodiments of the present disclosure. The ultrasound device 10 includes an ultrasound probe 12 that, in the illustrated embodiment, is electrically coupled to a handheld computing device 14 by a cable 17. The cable 17 includes a connector 18 that detachably connects the probe 12 to the computing device 14. The handheld computing device 14 may be any portable computing device having a display, such as a tablet computer, a smartphone, or the like. In some embodiments, the ultrasound probe 12 need not be electrically coupled to the handheld computing device 14, but may operate independently of the handheld computing device 14, possibly communicating with the handheld computing device 14 via a wireless communication channel.

The probe 12 is configured to transmit an ultrasound signal toward a target structure and to receive echo signals returning from the target structure in response to transmission of the ultrasound signal. As illustrated, the probe 12 includes a transducer array 20 including transducer elements that are capable of transmitting an ultrasound signal and receiving subsequent echo signals.

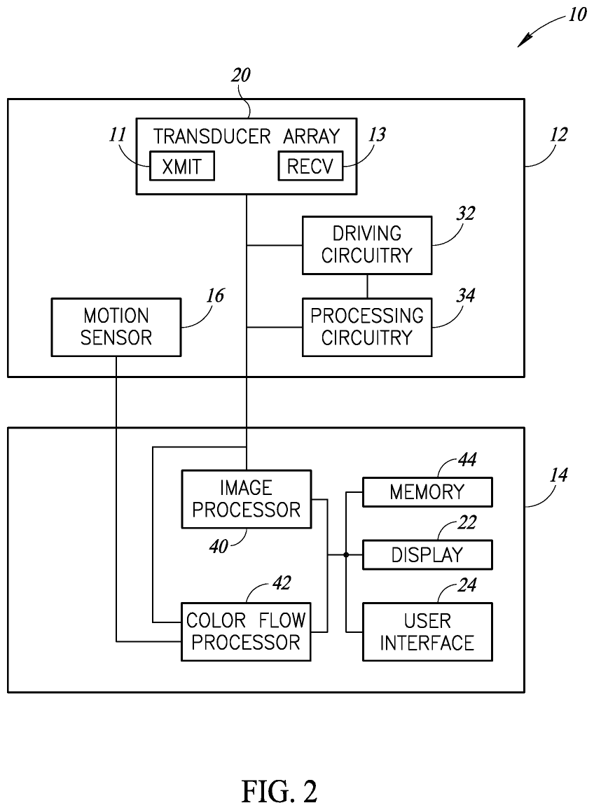

As will be described in greater detail in connection with FIG. 2, the ultrasound device 10 further includes processing circuitry and driving circuitry. In part, the processing circuitry controls the transmission of the ultrasound signal from the transducer array 20. The driving circuitry is operatively coupled to the transducer array 20 for driving the transmission of the ultrasound signal, e.g., in response to a control signal received from the processing circuitry. The driving circuitry and processor circuitry may be included in one or both of the ultrasound probe 12 and the handheld computing device 14. The ultrasound device 10 also includes a power supply that provides power to the driving circuitry for transmission of the ultrasound signal, for example, in a pulsed wave or a continuous wave mode of operation.

The transducer array 20 of the probe may include one or more transmit transducer elements 11 that transmit the ultrasound signal and one or more receive transducer elements 13 that receive echo signals returning from a target structure in response to transmission of the ultrasound signal. In some embodiments, some or all of the transducer elements of the transducer array 20 may act as transmit transducer elements 11 during a first period of time and as receive transducer elements 13 during a second period of time that is different than the first period of time (i.e., the same transducer elements may be usable to transmit the ultrasound signal and to receive echo signals at different times).

The probe 12 further includes a motion sensor 16, which is operable to sense a motion of the probe 12. The motion sensor 16 is included in or on the probe 12 and may include, for example, one or more accelerometers or gyroscopes for sensing motion of the probe 12. For example, the motion sensor 16 may be or include any of a piezoelectric, piezoresistive or capacitive accelerometer capable of sensing motion of the probe 12. In some embodiments, the motion sensor 16 may be a tri-axial motion sensor capable of sensing motion about any of three axes. In some embodiments, more than one motion sensor 16 is included in or on the probe 12. In some embodiments, the motion sensor 16 includes at least one accelerometer and at least one gyroscope.

The motion sensor 16 may be housed at least partially within an outer housing of the probe 12. In some embodiments, the motion sensor 16 is positioned at or near a sensing surface 21 of the probe 12. In some embodiments, the sensing surface 21 is a surface which is operably brought into contact with a patient during ultrasonic imaging. The transducer array 20 is positioned at or near the sensing surface 21 as shown in FIG. 1. In some embodiments, one or more additional sensors are positioned at or near the sensing surface 21, including, for example, one or more electrocardiography (EKG) sensors or electrodes, one or more auscultation sensors, or the like.

By positioning the motion sensor 16 near the sensing surface 21 of the probe 12, and hence near the transducer array 20, the motion sensor 16 may sense the motions of the sensing surface 21 (and transducer array 20) as it is used, for example, during ultrasound imaging.

The computing device 14 shown in FIG. 1 includes a display screen 22 and a user interface 24. The display screen 22 may be a display incorporating any type of display technology including, but not limited to, LED display technology. The display screen 22 is used to display one or more images generated from echo data obtained from the echo signals received in response to transmission of an ultrasound signal, and in some embodiments, the display screen 22 may be used to display color flow image information, for example, as may be provided in a Color Doppler imaging (CDI) mode. In some embodiments, the display screen 22 may be a touch screen capable of receiving input from a user that touches the screen. In such embodiments, the user interface 24 may include a portion or the entire display screen 22, which is capable of receiving user input via touch. In some embodiments, the user interface 24 may include one or more buttons, knobs, switches, and the like, capable of receiving input from a user of the ultrasound device 10. In some embodiments, the user interface 24 may include a microphone 30 capable of receiving audible input, such as voice commands.

The computing device 14 may further include one or more audio speakers 28 that may be used to generate audible representations of echo signals or other features derived from operation of the ultrasound device 10.

FIG. 2 is a block diagram illustrating components of the ultrasound device 10, including the ultrasound probe 12 and the computing device 14, in some embodiments. As shown in FIG. 2, the ultrasound device 10 may include driving circuitry 32 and processing circuitry 34 for controlling and driving the transmission of an ultrasound signal from the transducer array 20 of the ultrasound probe 12. The driving circuitry 32 and processing circuitry 34 may be included in the probe 12 and/or the computing device 14. In some embodiments, one or both of the driving circuitry 32 and processing circuitry 34 are included in the ultrasound probe 12. That is, the ultrasound probe 12 may contain the circuitry that controls the driving the transducer array 20 to transmit an ultrasound signal, and may further include circuitry for processing received echo signals.

In various embodiments, the processing circuitry 34 includes one or more programmed processors that operate in accordance with computer-executable instructions that, in response to execution, cause the programmed processor(s) to perform various actions. For example, the processing circuitry 34 may be configured to send one or more control signals to the driving circuitry 32 to control the transmission of an ultrasound signal by the transducer array 20 of the ultrasound probe 12.

The driving circuitry 32 may include an oscillator or other circuitry that is used when generating an ultrasound signal to be transmitted by the transducer array 20. Such an oscillator or other circuitry may be used by the driving circuitry 32 to generate and shape the ultrasound pulses that form the ultrasound signal.

The computing device 14 further includes an image processor 40 that processes received ultrasound image information (e.g., ultrasound information received from the receive transducer elements 13 and/or from the processing circuitry 34) and generates ultrasound image information for display on the display 22. In some embodiments, the image processor 40 generates ultrasound image information for B-mode imaging. In some embodiments, the image processor 40 is operable to generate ultrasound image information for any ultrasound imaging modality, including, for example, A-mode, C-mode, Doppler, or the like.

The computing device 14 further includes a color flow processor 42. The color flow processor 42 processes color flow information during color flow imaging, such as in a CDI mode. The color flow processor 42 may be any processor or processing circuitry operable to determine or estimate color flow information based on the received ultrasound information (e.g., ultrasound information received from the receive transducer elements 13 and/or from the processing circuitry 34), such as color flow information indicative of a blood or fluid flow velocity. The color flow processor 42 may generate the color flow information based on the received ultrasound information.

Each of the image processor 40 and the color flow processor 42 may include one or more programmed processors that operate in accordance with computer-executable instructions that, in response to execution, cause the programmed processor(s) to perform various actions as described herein. In some embodiments, the image processor 40 and/or the color flow processor 42 may be a programmed processor and/or an application specific integrated circuit configured to provide the image and color flow processing functions described herein.

The color flow information generated by the color flow processor 42 may be combined with or otherwise associated with the ultrasound image information generated by the image processor 40 to produce color flow image information which may be provided to the display 22 for displaying an ultrasound color flow image. For example, the ultrasound color flow image may include the color flow information generated by the color flow processor 42 overlaid or superimposed on B-mode image information generated by the image processor 40.

The color flow processor 42 is further configured to control or adjust one or more parameters of the displayed ultrasound color flow image, as will be discussed in further detail herein. The image processor 40 and/or the color flow processor 42 may be coupled to computer-readable memory 44, which may store computer-executable instructions that, in part, are executable by the image processor 40 and/or the color flow processor 42 and cause the image processor 40 and/or the color flow processor 42 to perform the various actions described herein.

The image processor 40 and the color flow processor 42 are coupled to the user interface 24. The user interface 24 may receive user input, for example, as touch inputs on the display 22, or as user input via one or more buttons, knobs, switches, and the like. In some embodiments, the user interface 24 may receive audible user input, such as voice commands received by a microphone 30 of the computing device 14. The image processor 40 and the color flow processor 42 are configured to provide the ultrasound color flow image information, and to control one or more parameters of the ultrasound color flow images displayed on the display 22, based on user input received by the user interface 24.

During operation of the ultrasound device 10, the ultrasound probe 12 acquires ultrasound signals, e.g., echo signals returning from the target structure in response to a transmitted ultrasound signal. The echo signals may be provided to the processing circuitry 34 and/or the image processor 40, either or both of which may include ultrasound image processing circuitry for generating ultrasound image information based on the received echo signals. Such ultrasound image processing circuitry may include, for example, amplifiers, analog-to-digital converters, delay circuitry, logic circuitry, and the like, which is configured to generate ultrasound image information based on the received echo signals.

The ultrasound image information is provided to the image processor 40, which generates or otherwise outputs ultrasound images associated with the received ultrasound signals to the display 22 for displaying the ultrasound images. Such ultrasound images may be ultrasound images associated with any of a variety of ultrasound imaging modes, such as A-mode (amplitude mode), B-mode (brightness mode), M-mode (motion mode), Doppler mode (including Color Doppler, Continuous Wave (CW) Doppler, and Pulsed Wave (PW) Doppler), and so on. Moreover, the ultrasound images may be 2D, 3D, or 4D ultrasound images.

Additionally, the echo signals or any processed information based on the received echo signals may be provided to the color flow processor 42 for estimating or determining color flow information. Such color flow information may include, for example, velocity and power (or energy) information.

The color flow processor 42 also receives motion information from the motion sensor 16. The motion information may be temporally correlated to the ultrasound image information, e.g., provided to the image processor 40. The color flow processor 42 may adjust, based on the received motion information, one or more color flow parameters associated with ultrasound color flow images provided for display on the display 22. For example, in some embodiments, the color flow processor 42 is configured to adjust a color (or velocity) flow threshold based on the motion information received from the motion sensor 16. A color flow threshold may be any threshold associated with a determined or estimated flow velocity. For example, the color flow threshold may be a low velocity threshold in which flow velocities below the color flow threshold are removed or otherwise suppressed from the ultrasound color flow image provided to the display 22. In some embodiments, the color flow threshold may be a high velocity threshold in which flow velocities above the color flow threshold are removed or otherwise suppressed from the ultrasound color flow image provided to the display 22. In some embodiments, the color flow threshold may be two or more thresholds, for example, including both a low velocity threshold and a high velocity threshold, such that the color flow threshold represents a range of velocities which may be displayed as color flow information in an ultrasound color flow image.

In some embodiments, the color flow parameters that are adjustable by the color flow processor 42 may include a power (or energy) threshold. In such embodiments, when the power or energy is below a low power threshold, or above a high power threshold, a flow associated with a particular spatial location associated with the low or high power information may be removed or suppressed from the ultrasound color flow image provided to the display 22.

In some embodiments, the color flow processor 42 may compare the motion information received from the motion sensor 16 to one or more motion thresholds, and may adjust one or more color flow parameters in response to the motion information exceeding the one or more motion thresholds. Such adjustments are typically temporary, e.g., for a period of time corresponding to when the received motion information exceeds the one or more motion thresholds. For example, in some embodiments, the color flow processor 42 compares motion information received from the motion sensor 16 to an acceleration threshold, and the color flow processor 42 may adjust a color flow parameter in response to the motion information indicating that the motion of the probe 12 exceeds the acceleration threshold. In such a case, the color flow processor 42 may, for example, increase the color flow velocity threshold so that motion artifacts, or spurious flow information caused by movement of the probe 12, are filtered out by the increased color flow velocity threshold (e.g., a low velocity threshold), and thus such motion artifacts are not present in the ultrasound color flow image provided to the display 22. By increasing the color flow velocity threshold, the color flow processor 42 can remove from the ultrasound color flow image motion artifacts that are caused, for example, by sudden or jerking movements of the ultrasound probe 12. Such motions of the probe 12 may otherwise create high velocity, high magnitude color information in the ultrasound color flow image that is not indicative of flow (such as blood flow) of interest. When the color flow processor 42 determines that the motion information no longer exceeds the one or more motion thresholds, the color flow processor 42 may cause the adjusted color flow parameter(s) to return to their previous, unadjusted value(s).

In some embodiments, the color flow processor 42 may compare motion information received from the motion sensor 16 to a probe velocity threshold. For example, if the probe 12 is moved smoothly (e.g., without excessive accelerations) but at a high velocity, this can result in unwanted motion artifacts being present in the ultrasound color flow images. Such motion artifacts can be removed by the color flow processor 42, for example, by dynamically increasing the color flow threshold in response to determining that the probe motion sensed by the motion sensor 16 exceeds the probe velocity threshold, thereby filtering out the motion artifacts associated with the high velocity motion of the probe 12.

The color flow parameters are accessible by the color flow processor 42 so that the color flow processor 42 may dynamically adjust the color flow parameters based on the motion information received from the motion sensor. In some embodiments, the color flow parameters or information associated with or otherwise defining the color flow parameters may be stored in the memory 44. Similarly, the one or more motion thresholds (or information associated with or defining the one or more motion thresholds) may be stored in the memory 44 and accessible by the color flow processor 42.

In some embodiments, different color flow parameters and/or the motion thresholds may be selected, for example, depending on a type of examination to be performed using the ultrasound device 10. Flow characteristics, such as flow velocity, may be different depending on the examination to be performed. For example, when imaging the liver, one is generally concerned with relatively slow or low velocity blood flows. On the other hand, if one is interested in turbulence in the heart, high velocity flows are of particular interest. The color flow processor 42 may access and use a particular set of color flow parameters based on a selected examination to be performed, which may be selected, for example, by a user input received via the user interface 24. As an example, a user may provide input via the user interface 24 which indicates that a liver examination is to be performed by the ultrasound device 10, and the color flow processor 42 may access and apply one or more color flow parameters associated with the liver examination, which may include, for example, a relatively low lower velocity threshold in order to retain color flow information associated with the relatively low velocity blood flows in the liver. Similarly, a user may indicate that a heart examination is to be performed, and the color flow processor 42 may access and apply one or more color flow parameters associated with a heart examination, which may include a relatively high upper velocity threshold in order to retain color flow information associated with the relatively high velocity blood flows in the heart.

Moreover, the one or more motion thresholds that are accessed by the color flow processor 42 and applied during use of the ultrasound device 10 may be selected based on a type of examination to be performed. For example, if the user indicates that a liver examination is to be performed, the color flow processor 42 may access and apply a relatively low motion threshold, since the signals of interest (e.g., blood flow in the liver) are indicative of relatively low velocity flows. That is, by selecting a relatively low motion threshold for a particular examination (such as a liver examination), the color flow processor 42 may be more sensitive to motion artifacts caused by user motions of the probe 12. Similarly, if the user indicates that a heart examination is to be performed, the color flow processor 42 may access and apply a relatively high motion threshold which will permit greater user motions of the probe 12, since the signals of interest are indicative of relatively high velocity flows.

In some embodiments, the motion sensor 16 includes two or more motion sensors, each of which may have an associated motion threshold. For example, in an embodiment, the probe 12 includes an accelerometer and a gyroscope. The motion information received from the accelerometer may be compared to an accelerometer motion threshold, and the motion information received from the gyroscope may be compared to a gyroscope motion threshold. The color flow processor 42 may adjust, based on the motion information received from either or both of the accelerometer and the gyroscope, one or more color flow parameters associated with ultrasound color flow images provided for display on the display 22.

For example, in some embodiments, the color flow processor 42 may adjust a color flow parameter in response to the motion information received from the accelerometer being greater than the accelerometer motion threshold, even if the motion information received from the gyroscope is below the gyroscope motion threshold. Similarly, in some embodiments, the color flow processor 42 may adjust a color flow parameter in response to the motion information received from the gyroscope being greater than the gyroscope motion threshold, even if the motion information received from the accelerometer is below the accelerometer motion threshold.

In some embodiments, the color flow processor 42 may adjust a color flow parameter only in response to motion information received from both the accelerometer and the gyroscope being greater than the accelerometer motion threshold and the gyroscope motion threshold, respectively.

In some embodiments, the color flow processor 42 may adjust any parameter associated with ultrasound color flow images to be displayed on the display 22, which are referred to herein as color flow parameters. For example, one such color flow parameter is a frame average or “persistence” parameter, which may be adjusted to change a smoothness and/or reduce noise in the displayed ultrasound images. For example, in response to motions of the probe 12 which exceed one or more motion thresholds, the color flow processor 42 may reduce a persistence parameter, which may reduce the persistence of color flow information associated with sudden or abrupt motions of the probe 12. Another color flow parameter which may be adjusted by the color flow processor 42 in response to motions of the probe 12 exceeding one or more motion thresholds is the color gain. Adjusting the color gain may change the sensitivity of the ultrasound imaging device 10 to flow signals. For example, increasing the color gain amplifies the appearance of flow in the acquired ultrasound signals, while decreasing the color gain will decrease the appearance of flow in the ultrasound signals. In some embodiments, the color flow processor 42 may reduce the color gain in response to motions of the probe 12 exceeding one or more motion thresholds, which will decrease the sensitivity of the ultrasound imaging device 10 to motion artifacts which may provide a false appearance of flow in the ultrasound color flow images.

In some embodiments, the motion sensor 16 may determine whether the motion information (e.g., the information indicative of a sensed motion of the probe 12) sensed by the motion sensor 16 exceeds a motion threshold. That is, in some embodiments, the motion sensor 16 itself, rather than the color flow processor 42, performs the motion thresholding operations described herein. In such embodiments, the motion sensor 16 may output the sensed motion information only if the sensed motion information does not exceed the motion threshold. For example, if the sensed motion is below the motion threshold, the motion information which indicates motion of the probe 12 is not provided to the color flow processor 42, in which case, the color flow processor 42 may generate color flow information without consideration of any movements of the probe 12, since such movements are below the motion threshold. On the other hand, if the motion sensor 16 determines that the sensed motion exceeds the motion threshold, then the motion information sensed by the motion sensor 16 is provided to the color flow processor 42, which may adjust one or more color flow parameters based on the motion information. The motion sensor 16 may include or otherwise access processing circuitry to perform a comparison between the sensed motion information and a motion threshold or to otherwise determine whether the sensed motion of the probe 12 exceeds a motion threshold.

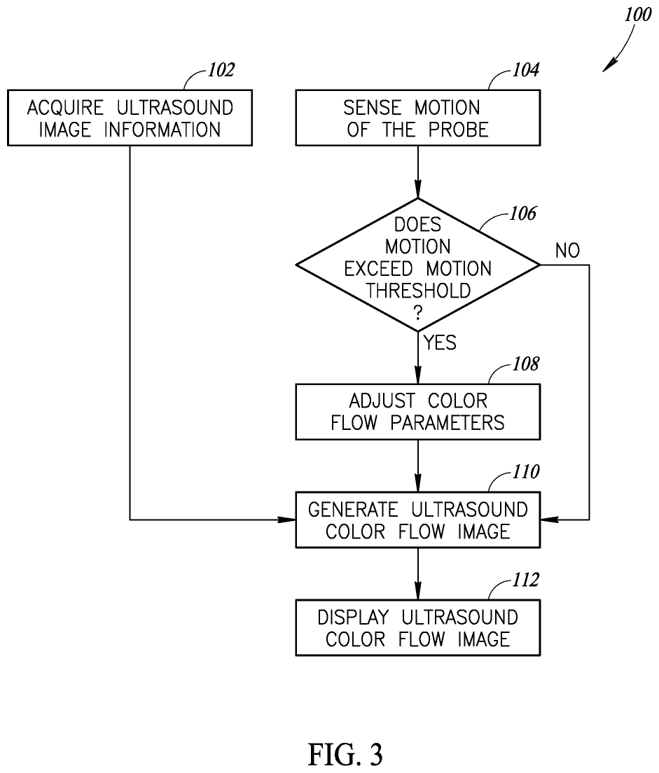

FIG. 3 is a flow diagram illustrating a method 100, in accordance with one or more embodiments of the present disclosure. In at least one embodiment, the method 100 includes acquiring ultrasound image information at 102. The ultrasound image information may be acquired, for example, by the ultrasound imaging device 10.

At 104, the method 100 includes sensing a motion of the probe 12 of the ultrasound imaging device 10. The motion of the probe 12 may be sensed, for example, by the motion sensor 16, which may generate motion information indicative of the sensed motion of the probe 12. In some embodiments, the motion information sensed by the motion sensor 16 may be temporally correlated with the acquired ultrasound image information, which may facilitate the generation of ultrasound color flow images that are motion-compensated (e.g., by adjusting one or more color flow parameters) based on the sensed motions of the probe 12 at the time that the corresponding ultrasound image information is acquired. In some embodiments, the motion of the probe 12 is sensed by an accelerometer, a gyroscope, or by both an accelerometer and a gyroscope. For example, the accelerometer may generate a first motion signal indicative of the motion of the ultrasound probe, and the gyroscope may generate a second motion signal indicative of the motion of the ultrasound probe.

At 106, the motion information sensed by the motion sensor 16 is compared to a motion threshold to determine whether the motion of the probe 12 exceeds the motion threshold. The determination of whether the motion of the probe 12 exceeds the motion threshold may be performed, for example, by the color flow processor 42, or by the motion sensor 16. In some embodiments, the motion information sensed by the motion sensor 16 is compared to multiple motion thresholds. For example, in embodiments where the motion sensor 16 includes both an accelerometer and a gyroscope, each of the accelerometer and the gyroscope may output respective sensed motion information which may be compared to separate motion thresholds to determine, for example, whether the first motion signal (e.g., generated by the accelerometer) exceeds a first motion threshold and whether the second motion signal (e.g., generated by the gyroscope) exceeds a second motion threshold.

At 108, one or more color flow parameters are adjusted in response to the motion of the probe 12 being determined as exceeding the motion threshold at 106. The one or more color flow parameters may be adjusted, for example, by the color flow processor 42 as previously described herein. In some embodiments in which the motion sensor 16 includes both an accelerometer and a gyroscope, the one or more color flow parameters may be adjusted in response to sensed motion information from each of the accelerometer and the gyroscope exceeding a respective motion threshold. In some embodiments, the one or more color flow parameters may be adjusted in response to motion information sensed by either the accelerometer or the gyroscope exceeding a respective motion threshold.

At 110, ultrasound color flow images are generated, for example, by the ultrasound imaging device 10. If at 106, the sensed motion of the probe 12 is determined to exceed the motion threshold, then the ultrasound color flow image is generated at 110 based on the acquired ultrasound image information and the adjusted one or more color flow parameters (e.g., as adjusted at 108). In such a case, motion artifacts due to the motions of the probe 12 are filtered from the generated ultrasound color flow image by the adjusted color flow parameters. On the other hand, if at 106 it is determined that the sensed motion of the probe 12 is below the motion threshold, then the ultrasound color flow image is generated based on the acquired ultrasound image information, without adjusting the color flow parameters.

At 112, the generated ultrasound color flow image is displayed, for example, on the display 22 of the computing device 14.

In some embodiments, the method 100 may further include receiving a selection of a type of examination to be performed by the ultrasound imaging device. For example, prior to acquiring the ultrasound image information (at 102) and sensing the motion of the probe (at 104), the ultrasound imaging device 10 may receive a selection of a type of examination to be performed. The selection may be received, for example, by a user input via the user interface 24. The user may select from among a plurality of different types of examinations to be performed, which may include ultrasound examinations to be performed with respect to different internal regions or organs of a patient, such as a liver examination, a heart examination, or any other ultrasound examination type.

In some embodiments, the method 100 may include accessing a color flow parameter associated with the selected type of examination to perform, and adjusting one or more color flow parameters (at 108) may include adjusting the accessed color flow parameter associated with the selected type of examination to perform. Moreover, generating an ultrasound color flow image (at 110) may include generating an ultrasound color flow image based on the acquired ultrasound image information and the accessed color flow parameter associated with the selected type of examination to perform.

In some embodiments, the method 100 may include accessing a motion threshold associated with the selected type of examination to perform, and determining whether the sensed motion of the ultrasound probe exceeds a motion threshold (at 106) may include determining whether the sensed motion of the ultrasound probe exceeds the accessed motion threshold associated with the selected type of examination to perform.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
acquiring, by an ultrasound imaging device including an ultrasound probe, ultrasound image information;
sensing, by a motion sensor, a motion of the ultrasound probe;
determining whether the sensed motion of the ultrasound probe exceeds a motion threshold;
adjusting one or more color flow parameters in response to determining that the sensed motion of the ultrasound probe exceeds the motion threshold; and
generating an ultrasound color flow image based on the acquired ultrasound image information and the one or more color flow parameters.

2. The method of claim 1, further comprising:
displaying the ultrasound color flow image on a display of a computing device.

3. The method of claim 1 wherein the sensing a motion of the ultrasound probe includes sensing a motion of the ultrasound probe by at least one of an accelerometer or a gyroscope.

4. The method of claim 1 wherein the adjusting one or more color flow parameters includes adjusting at least one of a color flow threshold or a power threshold.

5. The method of claim 4 wherein the adjusting one or more color flow parameters includes increasing the color flow threshold.

6. The method of claim 1 wherein the sensing a motion of an ultrasound probe includes:
generating by an accelerometer a first motion signal indicative of the motion of the ultrasound probe; and
generating by a gyroscope a second motion signal indicative of the motion of the ultrasound probe.

7. The method of claim 6 wherein the determining whether the sensed motion of the ultrasound probe exceeds a motion threshold includes:
determining whether the first motion signal exceeds a first motion threshold; and
determining whether the second motion signal exceeds a second motion threshold.

8. The method of claim 1 wherein the generating an ultrasound color flow image based on the acquired ultrasound image information and the one or more color flow parameters includes:
removing, based on the one or more color flow parameters, motion artifacts from the ultrasound color flow image.

9. The method of claim 1, further comprising:
receiving a selection of a type of examination to be performed by the ultrasound imaging device; and
accessing a color flow parameter associated with the selected type of examination to perform,
wherein the adjusting one or more color flow parameters includes adjusting the accessed color flow parameter associated with the selected type of examination to perform, and
wherein the generating an ultrasound color flow image includes generating an ultrasound color flow image based on the acquired ultrasound image information and the accessed color flow parameter associated with the selected type of examination to perform.

10. The method of claim 1, further comprising:
receiving a selection of a type of examination to be performed by the ultrasound imaging device; and
accessing a motion threshold associated with the selected type of examination to perform,
wherein the determining whether the sensed motion of the ultrasound probe exceeds a motion threshold includes determining whether the sensed motion of the ultrasound probe exceeds the accessed motion threshold associated with the selected type of examination to perform.

11. An ultrasound imaging device, comprising:
an ultrasound probe, the ultrasound probe including at least one motion sensor which, in use, senses motion of the ultrasound probe;
a computer-readable memory storing color flow parameters; and
a color flow processor coupled to the motion sensor, wherein the color flow processor, in use:
receives ultrasound image information acquired by the ultrasound probe;
accesses at least one of the color flow parameters stored in the computer-readable memory;
receives, from the at least one motion sensor, motion information indicative of a sensed motion of the ultrasound probe;
adjusts the at least one of the color flow parameters in response to the sensed motion of the ultrasound probe exceeding a motion threshold; and
generates an ultrasound image based on the ultrasound image information and the at least one of the color flow parameters.

12. The ultrasound imaging device of claim 11, further comprising:
a display coupled to the color flow processor, wherein the display, in use, displays the generated ultrasound color flow image.

13. The ultrasound imaging device of claim 11 wherein the at least one motion sensor includes at least one of an accelerometer or a gyroscope.

14. The ultrasound imaging device of claim 11 wherein the at least one motion sensor includes an accelerometer and a gyroscope.

15. The ultrasound imaging device of claim 11 wherein the color flow processor, in use:
determines whether the sensed motion of the ultrasound probe exceeds the motion threshold.

16. The ultrasound imaging device of claim 11 wherein the at least one motion sensor, in use:
determines whether the sensed motion of the ultrasound probe exceeds the motion threshold; and
outputs the motion information indicative of the sensed motion of the ultrasound probe in response to determining that the sensed motion of the ultrasound probe exceeds the motion threshold.

17. A system, comprising:
an ultrasound probe configured to acquire ultrasound image information;
at least one motion sensor coupled to the ultrasound probe, wherein the at least one motion sensor is configured to sense a motion of the ultrasound probe; and
a computing device coupled to the ultrasound probe and to the at least one motion sensor, the computing device including a color flow processor configured to:
determine whether the sensed motion of the ultrasound probe exceeds a motion threshold;
adjust one or more color flow parameters in response to determining that the sensed motion of the ultrasound probe exceeds the motion threshold; and
generate an ultrasound color flow image based on the acquired ultrasound image information and the one or more color flow parameters.

18. The system of claim 17 wherein the computing device includes a display configured to display the generated ultrasound color flow image.

19. The system of claim 17 wherein the computing device includes a computer-readable memory configured to store the one or more color flow parameters and the motion threshold.

20. The system of claim 17 wherein the at least one motion sensor includes an accelerometer and a gyroscope.
